(19) United States
(12) Patent Application Publication
(10) Pub. No.: US 2025/0359930 A1
(43) Pub. Date: Nov. 27, 2025

(54) MICROWAVE-BASED NON-INVASIVE SKIN TIGHTENING SYSTEMS AND METHODS

(71) Applicant: Cynosure, LLC, Westford, MA (US)

(72) Inventors: Bo Chen, Los Altos, CA (US); James Boll, Auburndale, MA (US); Donald K. Smith, Newton, MA (US); Daniel Smith, Boston, MA (US)

(73) Assignee: Cynosure, LLC, Westford, MA (US)

(21) Appl. No.: 19/203,824

(22) Filed: May 9, 2025

Related U.S. Application Data
(60) Provisional application No. 63/651,122, filed on May 23, 2024.

(51) Int. Cl. A61B 18/18; A61B 18/00

(52) U.S. Cl. CPC A61B 18/1815; A61B 2018/0047; A61B 2018/00589; A61B 2018/1823; A61B 2018/183; A61B 2018/1876

(57) ABSTRACT

In part, in one aspect, the disclosure relates to a non-invasive skin tightening system. The system may include an applicator comprising a first surface, the first surface defining one or more apertures; a waveguide in communication with the first surface and positioned to direct microwaves through the one or more apertures; and a microwave generator having a peak power, a pulse width, and a microwave frequency. The applicator is a cosmetic treatment applicator in various embodiments.

### CROSS REFERENCE TO RELATED APPLICATIONS

This application claims priority to and the benefit of U.S. Provisional Patent Application No. 63/651,122 entitled “Microwave-based Non-invasive Skin Tightening Systems and Methods” filed on May 23, 2024, the disclosure of which is herein incorporated by reference in its entirety.

### FIELD

This disclosure relates generally to the field of non-invasive microwave-based cosmetic treatments and related systems.

### BACKGROUND

Skin laxity is a major feature of aging that until recently could only be addressed with surgery. Although dramatic clinical improvement can be achieved with surgical procedures, there are considerable associated side effects, postoperative recovery and monetary expense. As such, noninvasive or minimally invasive procedures with quick postoperative recovery and a low side-effect profile are in considerable demand. Historically, ablative and non-ablative laser devices have been used to improve facial skin laxity. Due to the limited penetration depth of light, most of the laser treatments can only target superficial layer of dermis, which limits the total volume of collagen the laser can affect. Recently novel technologies like Radiofrequency (RF) and High-Intensity Focused Ultrasound (HIFU) have emerged that use energy sources other than light and laser. Similar to laser and light energy, RF energy interacts with skin to induce thermal changes. The non-specific heating profile generated by non-invasive RF creates bulk heating and not ideal to create localized collagen coagulation. Intense focused ultrasound (IFUS) has been investigated to create localized coagulation zone in the deep dermis. The treatment usually is perceived to be very painful and requires sedation. The ratio of coagulation volume to the deep dermis is small due to its fractionated nature of delivery. Therefore, the amount of neocollagenesis and neoelastoegensis is limited. The present disclosure addresses some of the foregoing challenges and others.

### SUMMARY

A novel means of applying microwave energy is described here whereby a special selection of microwave characteristics (waveform, frequency, system design etc.) that match the physical and dielectric properties of targeted tissue can be employed to maximize a constructive interference pattern at the interface or junction of two or more tissues in a body region thereby creating a concentrated high energy zone in the tissues in the body region. The special selection of microwave characteristics including waveform, frequency, system design, etc. can be employed using microwave energy in propagation mode to target skin/fat junction or interface, also referred to as the dermal tissue and fat tissue junction or interface, or the dermal tissue and adipose tissue junction or interface. Due to the concentrated energy distribution achieved using the special selection of microwave characteristics, the size of tissue therapeutic heating can be limited to a sub-mm range, which is significantly smaller than a typical microwave tissue heating application. The selective heating of dermis at skin/fat junction with such a design can be employed in clinical application such as tissue tightening through applying microwave in propagation mode (although limited). Applying microwave in propagation mode requires a certain level of clinician skill to ensure that the energy is deposited at the skin/fat junction and, for example, away from areas where boney structures are close to the skin surface. Otherwise, safety and/or patient comfort may be compromised. Because the special selection of microwave characteristics ensures a concentrated energy distribution (e.g., in the sub-mm range) the skilled clinician can employ a propagating microwave system applicator with precision to ensure effective and safe treatment. In part, the disclosure relates to systems and methods of cosmetic tissue treatment, including for example, systems and methods of microwave-based cosmetic systems and methods.

This novel means of applying microwave energy is described here whereby the microwave propagation from the waveguide is designed to emit evanescent modes rather than propagating modes. Propagating modes are oscillating electrical and/or magnetic fields that propagate in free space. Evanescent modes are an oscillating electric and/or magnetic field that does not propagate as an electromagnetic wave but whose energy is spatially concentrated in the vicinity of the source. Since the evanescent mode microwave energy is spatially constrained to the output of an applicator waveguide in contact with the skin and water cooled to protect the skin, this energy can be efficiently coupled to the target tissue. Here the target tissue is defined as the interface between the dermis and the underlying adipose tissue where the objective is to non-invasively heat the dermis to a set temperature to induce changes such as neocollagenesis and neoclastoegensis, but leave the surrounding tissues unaffected. The interface of the two tissue planes, dermis and adipose tissue, represents a discontinuity of the tissue's dielectric properties where absorption of the evanescent microwave energy takes place. This is especially effective because the depth of this interface can vary, but the energy deposition and heating will always follow the anatomy and seek deposition at the interface of the skin tissue and fat tissue, and the deposition will occur regardless of skin thickness. Thus, whether the region of treatment has relatively thick skin (e.g., thighs) or relatively thin skin (e.g., neck) the disclosed treatment will heat only the treatment area (e.g., the interface of the skin tissue and fat tissue (S/F), the interface of the dermal tissue and fat tissue (D/F), the interface of the dermal tissue and adipose tissue (D/A), which are all the same area of treatment).

If propagating microwaves are used, this interface also generates reflections of the microwave energy that can be directed away from the applicator and heat other non-target tissues creating a safety risk. However, the properties of evanescent microwaves eliminate these reflections and concentrate the energy only to the target tissue interface. The device described here is a microwave generator and skin applicator that targets the deep (˜2 mm below the skin) dermal-adipose tissue plane and, as a further safety benefit, does not affect surrounding anatomical structures. Surrounding anatomical structures are not affected, because employing evanescent microwaves results in confinement of the energy and the risk of microwave energy being directed away from the applicator to non-target anatomical structures is avoided by the employing evanescent waves to treat target tissue.

In part, in one aspect, the disclosure relates to a non-invasive skin tightening system. The system may include an applicator comprising a first surface, the first surface defining one or more apertures; a waveguide in communication with the first surface and positioned to direct microwaves through the one or more apertures; and a microwave generator having a peak power, a pulse width, and a microwave frequency.

In some embodiments, the microwave frequency ranges from about 300 MHz to about 10 GHz. In some embodiments, the microwave frequency ranges from about 2 GHz to about 6 GHz. In some embodiments, the microwave frequency is about 5.8 GHz. In some embodiments, the one or more apertures is a slit, wherein slit has a length and a width. In some embodiments, the length ranges from about 5 mm to about 15 mm and wherein the width ranges from about 12.5 mm to about 18 mm. In some embodiments, the length of waveguide ranges from about 28 mm to about 38 mm, wherein width of waveguide ranges from about 12.5 mm to about 18 mm, and wherein height of waveguide is fixed to an integer multiple of half wavelengths. In some embodiments, the peak power range is greater than or equal to about 1 kW. In some embodiments, the peak power ranges from about 0.5 kW to about 10 KW. In some embodiments, the pulse width ranges from about 0.5 ms to about 100 ms. In various embodiments, the system may further include a control system, wherein the control system is in electrical communication with the microwave generator, wherein the control system is operable to cause the microwave generator to generate propagating microwaves, evanescent microwaves, and combinations thereof. In some embodiments, control system is operable to generate constructive interference of microwaves from the microwave generator at dermal and fat junction.

In some embodiments, the control system is operable to generate one or more confined thermal heating zones near the dermal and fat junction at one or more target tissue regions. In some embodiments, the one or more apertures comprising differing or repeating shapes in various combinations that are suitable for supporting propagating or evanescent microwaves. In some embodiments, the one or more apertures comprise one or more shapes configured to support propagating or evanescent microwaves. In some embodiments, control system regulates the microwave generator to cause localized heating that can result in collagen coagulation at one or more target tissue regions. In some embodiments, the microwave generator has a peak power that ranges from about 1 kW to about 20 KW. In various embodiments, the integer multiple of half wavelengths is about N (25.85) mm, wherein N is a positive integer. In some embodiments, the waveguide has an internal dimension that includes one or more of a height of about 34.8 mm, a width of about 25.8 mm and a depth of about 15.8 mm.

In part, in another aspect, the disclosure relates to a non-invasive skin tightening system. The system may include an applicator comprising a first surface, the first surface defining one or more apertures; a waveguide in communication with the first surface and positioned to generate evanescent waves; and a microwave generator having a peak power, a pulse width, and a microwave frequency.

In some embodiments, the waveguide has an internal dimension that includes one or more of a height of about 34.8 mm, a width of about 25.8 mm and a depth of about 15.8 mm. In some embodiments, the system may further include a cooling system in communication with a treatment surface of the applicator and or the microwave generator or waveguide. In some embodiments, the applicator is a handheld device comprising a first surface, a housing, and an umbilical, wherein the umbilical is operably coupled to the microwave generator. In some embodiments, microwave frequency ranges from about 300 MHz to about 10 GHz. In some embodiments, the microwave frequency ranges from about 2 GHz to about 6 GHz. In some embodiments, the peak power has a range, wherein the range is greater than or equal to about 1 kW. In some embodiments, the peak power ranges from about 0.5 kW to about 10 kW. In some embodiments, the pulse width ranges from about 0.5 ms to about 100 ms.

In part, in yet another aspect, the disclosure relates to a non-invasive method for tightening skin. The method may include applying to a skin surface an applicator surface defining one or more apertures; and generating evanescent waves with a waveguide in communication with the applicator surface, the waveguide emitting a microwave having a peak power, a pulse width, and a microwave frequency.

In some embodiments, the method may further include cooling a treatment surface of the applicator and/or the microwave generator or waveguide. In some embodiments, the waveguide has internal dimension that include one or more of a height of about 34.8 mm, a width of about 25.8 mm and a depth of about 15.8 mm. In some embodiments, the applicator is a handheld device comprising a first surface, a housing, and an umbilical, wherein the umbilical is operably coupled to the microwave generator. In some embodiments, microwave frequency ranges from about 300 MHz to about 10 GHz. In some embodiments, the microwave frequency ranges from about 2 GHZ to about 6 GHz. In some embodiments, the peak power range is greater than or equal to about 1 kW. In some embodiments, the peak power ranges from about 0.5 kW to about 10 KW. In some embodiments, the pulse width ranges from about 0.5 ms to about 100 ms.

Although, the disclosure relates to different aspects and embodiments, it is understood that the different aspects and embodiments disclosed herein can be integrated, combined, or used together as a combination system, or in part, as separate components, devices, and systems, as appropriate. Thus, each embodiment disclosed herein can be incorporated in each of the aspects to varying degrees as appropriate for a given implementation. Further, the various systems, probes, applicators, arrays, waveguides, controllers, components and parts of the foregoing can be used with any suitable tissue surface, cosmetic and aesthetic applications, therapeutic applications, and medical applications and other methods and conjunction with other devices and systems without limitation.

These and other features of the applicant's teachings are set forth herein.

### BRIEF DESCRIPTION OF THE FIGURES

The figures are not necessarily to scale, emphasis instead generally being placed upon illustrative principles. The figures are to be considered illustrative in all aspects and are not intended to limit the disclosure, the scope of which is defined only by the claims.

FIG. 1 is a graph showing a relationship between conductivity and treatment energy frequency for adipose tissue versus skin tissue according to an illustrative embodiment of the disclosure.

FIG. 2 is a graph showing a relationship between relative permittivity and treatment energy frequency for adipose tissue versus skin tissue according to an illustrative embodiment of the disclosure.

FIG. 3 is a diagram that depicts various measurements to evaluate a waveguide design for a treatment energy generating apparatus relative to various parameters for skin, fat, and muscle tissue according to an illustrative embodiment of the disclosure.

FIG. 4 is a diagram that depicts specific absorption rates (SAR) in the skin and fat region when using treatment energy at two different frequencies according to an illustrative embodiment of the disclosure.

FIG. 5 is a plot that depicts SAR vs. distance from the skin surface using treatment energy at two different frequencies according to an illustrative embodiment of the disclosure.

FIG. 6 is a plot that depicts variability in the SAR at the skin/fat interface for different fat thicknesses using treatment energy according to an illustrative embodiment of the disclosure.

FIG. 7 shows two plots of SAR for the evanescent and propagating modes of treatment energy for the same total input power according to an illustrative embodiment of the disclosure.

FIG. 8 is a plot showing power dissipation of treatment energy at the center of the waveguide aperture according to an illustrative embodiment of the disclosure.

FIG. 9 is a plot showing temperature on the skin/fat interface at the center of the applicator for 1 kW of power input according to an illustrative embodiment of the disclosure.

FIG. 10 shows two plots of the temperature distribution in the skin, fat and muscle 10 seconds after a 60 ms application of 1 kW for evanescent and propagating treatment microwaves according to an illustrative embodiment of the disclosure.

FIG. 11 is a block diagram showing the path of the microwave energy from its source (the generator) to its ultimate destination (the patient) according to an illustrative embodiment of the disclosure.

FIG. 12 is a diagram showing the waveguide, and the electric field distribution produced by the microwaves with oscillating waves within the waveguide according to an illustrative embodiment of the disclosure.

FIGS. 13-16 depict various components of a microwave handpiece according to an illustrative embodiment of the disclosure.

FIG. 17 shows four waveguide endfaces suitable for generating treatment energy with different apertures according to an illustrative embodiment of the disclosure.

### DETAILED DESCRIPTION

In part, the disclosure relates to systems, devices, and methods of directing and/or delivering microwave) energy to one or more tissue regions, volumes, or layers to transform the foregoing tissue by one or more mechanisms of action such that cosmetic, rejuvenating, and/or other tissue changes directly or indirectly result or are initiated.

Microwaves are a form of electromagnetic radiation with wavelengths ranging from one meter to one millimeter corresponding to frequencies between 300 MHz and 300 GHz respectively. Each of the specific bands of electromagnetic and acoustic radiation, optical, RF, HFUS and MW, offer unique interactions and specificity within the tissue. Microwave has a deeper penetration depth in human tissue as compared to light. Therefore, microwave-based therapeutic medical devices generally are designed to heat a large volume (mm to cm in size) of tissue uniformly inside the human body. Output from these devices forms a propagating wave to deliver energy over a long distance. To achieve uniform heating over a large area (bulk heating), treatment time is long enough (typically in seconds to minutes) to allow 1) sufficient energy delivery and 2) some level of heat conduction in tissue for improved uniformity. In part, the disclosure relates to systems and methods of cosmetic tissue treatment, including for example, systems and methods of microwave-based cosmetic treatment systems and methods. In many embodiments, the systems and methods are non-invasive and non-surgical.

A waveguide is generally used to direct microwaves with minimal loss in a desired direction. Microwave propagation from the waveguide can be in propagating or evanescent modes. Propagating modes are oscillating electrical and/or magnetic fields that propagate in free space. Evanescent is an oscillating electric and/or magnetic field that does not propagate as an electromagnetic wave but whose energy is spatially concentrated in the vicinity of the source.

Microwaves penetrate deeply into tissue and can be designed to heat specific regions due to the dielectric property mismatch of different tissue planes. While the wavelengths are comparatively large, the heating can be confined due to the energy concentration at the tissue junction. By using the natural tissue planes to define the exact placement of the heat, the treatment is consistent for all tissue thickness variations on one patient or between patients.

A special selection of microwave characteristics (waveform, frequency, system design etc.) that match the physical and dielectric properties of targeted tissue can be made to maximize a constructive interference pattern at the interface or junction of two or more tissues in a body region (e.g., skin/fat junction or skin/fat interface), therefore creating a concentrated high energy zone at the junction. Due to the concentrated energy distribution, the size of tissue therapeutic heating can be limited to a sub-mm range, which is significantly smaller than a typical microwave tissue heating application.

A normal propagating microwave would still carry energy along a path and centimeters into the tissue and be subject to reflecting off another interface that was not intended (e.g. muscle/bone) thus presenting a safety concern. As microwaves propagate through the tissue, a significant amount of microwave energy can be reflected back from the dermal/fat interface due to the dielectrical property mismatch at the junction.

Additionally, the unique properties of the evanescent waves are novel to this application because the properties allow for the efficient deposition of microwave energy at the terminal end of the waveguide, for example, at the dermal fat interface, any residual energy not deposited at the dermal fat interface is substantially and rapidly attenuated. The rapid dissipation of energy after the terminal end of the waveguide (e.g., the target tissue) avoids unwanted collateral tissue damage. Thus, unlike a propagating wave that has directionality and maintains energy along its path, the evanescent wave is efficiently and safely dissipated into adjacent tissue causing no further energy concentration except where desired.

Additionally, evanescent microwave fields provide a rapid loss of or dissipation of energy near the exit aperture of the microwave delivery (for example, a waveguide). Usually, this energy propagation mode that dissipates quickly is considered inefficient. The rapid energy dissipation associated with evanescent microwave fields is typically undesirable for tissue treatment due to its limited depth of energy distribution.

In the application of skin tightening, an evanescent wave design coupled with the other microwave characteristics described above can further increase the concentration of microwave energy to the target area. The increased efficiency of energy in the focal zone also helps heat the target area in a short pulse before heat diffuses out the target and causes undesired side effects (e.g. pain, scar etc.)

The target region for skin tightening is the interface between skin tissue and fat tissue (e.g., the S/F interface). Special design considerations can be made to concentrate microwave energy at the interface of different tissues which have mismatches in dielectric properties (relative permittivity and microwave conductivity), regardless of tissue thickness. Thus, specially designed evanescent microwave output can adjust to the anatomy being treated regardless of whether the skin tissue is thick or thin. This technical approach is accordingly versatile for treating all regions of skin regardless of thickness, e.g., from the thick skin on the thighs to the thin skin on areas of the face, for example.

Laser energy applied to human tissue searches for and is selective for certain chromophores such that the laser matches the specific wavelength of light in the tissue to heat the selected tissue and this phenomenon is referred to as selective photothermolysis. The pulse width is shorter than the thermal relaxation time to create efficient heating of the target and avoid collateral damage. In an analogous way, this disclosure describes a selective microwave-thermolysis method using a novel microwave design to selectively heat the interface between two tissue types (e.g. S/F interface).

More specifically, microwave-based therapeutic medical devices generally include one or more antennas that convert high frequency currents (e.g., produced by a microwave generator) into a propagating electromagnetic wave. This wave may be transmitted from an applicator into one or more tissues of the body to produce a desired absorption pattern in tissue for targeted heating. In various embodiments, microwaves are delivered by an applicator that includes a waveguide with one or more tissue facing apertures. In various embodiments, the microwaves directed to various tissues promote, support or cause collagen coagulation via localized heating of tissue. In turn, in various embodiments, collagen coagulation results in tissue tightening such as skin tightening.

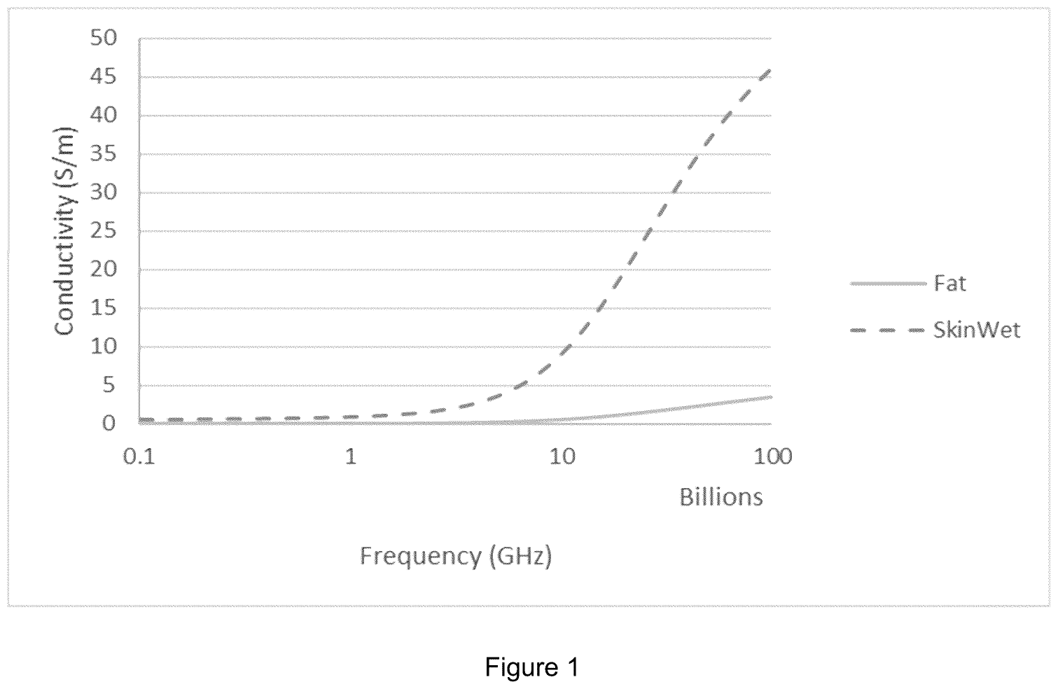

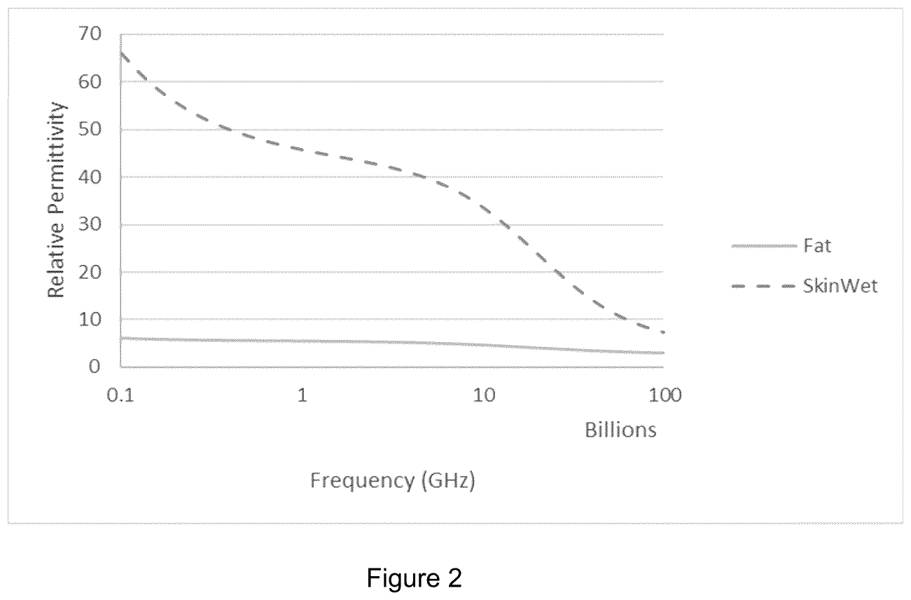

The absorption of microwave energy in tissue may initiate or cause dielectric heating in target tissue. Dielectric heating is a different way to generate heat rather than RF-based heating devices that cause heating via resistance to the flow of free electrons. In human tissue, microwave dielectric heating may be achieved by a rapidly changing electric field induced by a microwave signal acting upon dipole moments primarily within the water molecules present in tissue. In terms of microwave interaction with skin and fat, there are significant differences in the dielectric properties of the dermis and underlying adipose tissue. The relative permittivity and conductivity are significantly higher in the skin than adipose tissue. (See FIG. 1 and FIG. 2, based upon data and models presented by S. Gabriel, R. W. Lau and C. Gabriel in “The dielectric properties of biological tissues” Parts II & III in Phys. Med. Biol. 41 (1996) and discussed at http://niremf.ifac.cnr.it/tissprop/htmlclic/htmlclic.php).

The large difference in microwave dielectric properties between fat and skin may be used to achieve preferential selective heating of dermis rather than underlying adipose tissue. In various body tissues, a significant amount of microwave energy can be reflected back from the dermal/fat interface due to the dielectrical property mismatch at the junction. This reflected microwave energy may also be used to contribute to tissue changes in dermis and other skin tissues. Collagen-induced changes may benefit from receiving initial microwaves from an applicator and also from receiving microwaves reflected from the dermal/fat junction (i.e., D/F unction).

A customized microwave energy waveform can be used to maximize this phenomenon and create a constructive interference pattern at the dermal/fat junction. Taking advantage of preferred absorption in dermis and enhanced energy distribution at D/F junction caused by the constructive interference pattern, localized selective heating of deep dermis can be achieved non-invasively by a microwave irradiation. This customized waveform may be transmitted by an applicator that includes a customized waveguide and also by controlling power, frequency, pulse duration, and other parameters of the waveguide, microwave generator, power supply, transmitted microwaves, and the overall tissue treatment system. In various embodiments, a high power and short pulse duration are desirable.

In order to avoid collateral damage (such as burns, skin ulcerations, nerve damage, and others) exposure time to microwave energy is a selectable/controllable parameter of the system. This supports localizing heating to a focused/target tissue area or region. In various embodiments, exposure time ranges from about 5 ms to about 150 ms. In some embodiments, exposure time ranges from about 0.5 ms to about 20 ms. In other embodiments, the exposure time ranges from about 0.5 ms to about 125 ms, from about 0.5 ms to about 80 ms, from about 0.5 ms to about 50 ms, or from about 5 ms to about 35 ms.

Concentrated heating that can result in a localized injury may also be created using a heating pattern or an injury pattern generated by an array of waveguides (e.g., multiple antennae in contrast to a single waveguide/antennae). One or more relatively small island(s) of coagulation zones in deep dermis can be created that are surrounded by healthy tissue, which will help accelerate the tissue healing or wound-healing process and prevent scar formation. One way to cover a relatively large treatment zone is to use an array of antennas. Each antenna (or a group of antennae) can output microwave energy independently from others. Each antenna (or a group of antennae) can create one or multiple relatively small islands of coagulation zones in deep dermis. The handpiece includes such an array of antenna that can scan through the array in a predefined sequence and timing. The distribution of the antennae in the array will vary based upon the size of the handpiece in contact with the treatment area. The handpiece size is determined in part based upon the frequency utilized by the system. The physical size of each antenna and the size of the handpiece will determine how close the antennae are to one another and how many antennae are present in a specific handpiece.

Another benefit of limiting exposure time to microwave energy is for pain management (for example, treatment exposure time below about 100 ms, ideally between about 1 ms to about 10 ms, from about 0.5 ms to about 10 ms, or from about 0.5 ms to about 20 ms. A “heat-pain threshold” may correspond to the lowest heat stimulation intensity that is characterized as painful by a patient. The heat-pain threshold temperature is affected by many factors, such as the size of stimulus (spatial summation), the duration of stimulus, and the frequency of stimulus (temporal summation).

Generally, a shorter duration of heat is more tolerable than a relatively longer duration of heat. In some embodiments, one pulse or a small cluster or group of pulses is delivered at one location before moving on to another location to deliver pulses. Selective pulse delivery and microwave treatment times may be regulated in time and by position to manage discomfort during a cosmetic treatment. In various embodiments, a control system may be used to control the various exposure times, wave properties, and other parameters and treatment options and settings disclosed herein.

In addition, because the high temperature is created at D/F junction, in some embodiments, the superficial skin layer, which contains many nerve endings, can be maintained at lower temperature, and thereby lessen or minimize pain. Cooling can be employed to increase patient comfort during the treatment as well. In some embodiments, cooling may be achieved through either heat convection by blowing cold air on the skin tissue surface or heat conduction with a cold surface in contact with the skin tissue surface. It can be applied as short pulses or continuous pulses. Cooling may be provided as part of the applicator or through separate cooling devices. Depending on a given implementation, cooling may be performed using a constant temperature and intensity, or dynamically changed prior to applying radiation, during radiation, post radiation and/or adjusted based on pain sensation pre/during/post the microwave radiation treatment. In one embodiment, a thin layer of thermally conductive material (ceramic, sapphire, diamond, etc.) is a good choice to cover the aperture since it will not significantly attenuate the microwaves and serve to cool the skin immediately adjacent to the aperture and physically cover the open aperture.

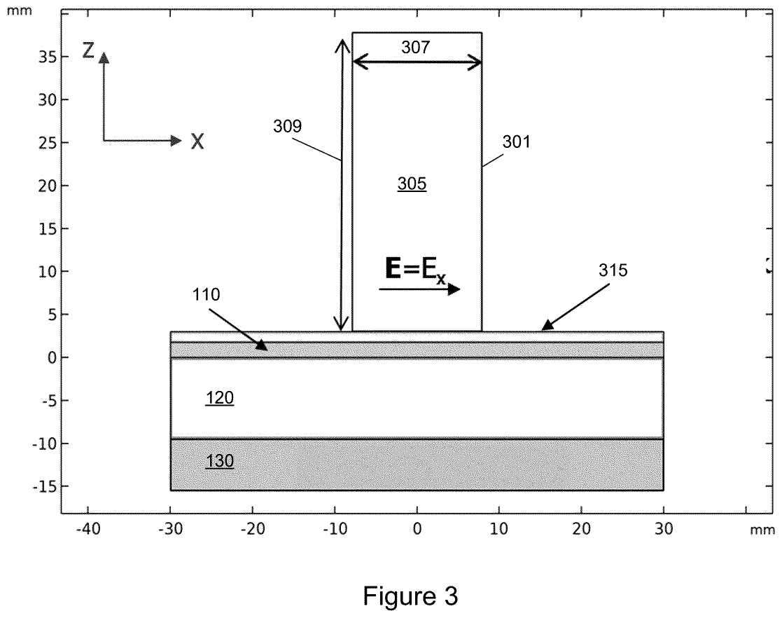

Various embodiments have been evaluated using 2D and 3D modeling of a prototype design using geometry and parameters shown in FIG. 3. In some embodiments, the microwave energy is fed into the top of the waveguide 305, and the wave propagates downwards, through a thin ceramic coating 315 into the skin 110, then fat 120, and finally muscle 130. In various embodiments, the width 307 of the waveguide 305 ranges from about 0.25 inches to about 2 inches. In some embodiments, the width 307 of the waveguide 305 is about 0.622 inches. In various embodiments, the length 309 of the waveguide 305 ranges from about 0.75 inches to about 2.25 inches. In some embodiments, the length 309 of the waveguide is about 1.372 inches. The width 307, length 309, and/or depth (not shown) of the waveguide 305 is selected to be large enough such that the applied frequency, nominally 5.8 GHz is higher than the cutoff frequency. For frequencies below the cutoff, the wave cannot propagate in the waveguide 305.

Standard waveguide configurations/dimensions exist which are appropriate for specific frequency bands. The WR137 waveguide, for example, has cross section dimensions of about 1.372 in (34.85 mm) by 0.622 in (15.8 mm), resulting in a cutoff frequency of 4.3 GHZ. Operating this waveguide at 5.8 GHz is comfortably above the cutoff frequency, making it a good off-the-shelf candidate, because waveguides employed in microwave energy applications filter out any frequency lower than the cutoff frequency. In this way, where the energy source operates at 5.8 GHz suitable waveguides have a cutoff frequency at 5.7 GHZ or lower. Thus, there will be many off the shelf or customized waveguides that are suited to frequency of the desired energy source, e.g., 5.8 GHZ.

The dimensions of the waveguide 305 (e.g., length 309 and width 307), the materials employed to create a waveguide, or other selected system (non-waveguide type of system) that can propagate a desired frequency are interdependent and are selected in concert to create the desired effect. For example, multiple waveguide dimensions could be employed based on a single desired frequency effect, likewise, multiple types of non-waveguide systems may be employed to achieve the same desired frequency.

FIG. 3 depicts an exemplary geometry suitable for some waveguide-based embodiments of the disclosure. In some embodiments, referring to FIG. 3, the thickness of the ceramic layer 315 on the waveguide 305 is about 1.25 mm thick. In various embodiments, the thickness of the ceramic layer 315 ranges from about 0.5 inches to about 3 inches. In some embodiments, the skin 110 is about 1.75 mm thick, although the thickness of the skin 110 may vary among different individuals and in different regions of the body. Fat 120 is below the skin 110 and in some embodiments the fat 120 is about 9.5 mm thick, although the thickness of the fat 120 may vary among different individuals and in different regions of the body. Further, muscle 130 is below the fat 120, which is below the skin 110 and the thickness of the muscle 130 may vary among different individuals and in different regions of the body and in one embodiment muscle 130 is about 6 mm thick.

In various embodiments, the waveguide 305 is an empty cavity defined by one or more conductive or semi-conductive surfaces. As a result, in some embodiments, there are minimal losses inside. Within the waveguide 305, the Specific Absorption Rate (SAR) is about zero in various embodiments. In some embodiments, there are also minimal losses in the ceramic layer 315. The ceramic layer 315 may include one or more materials having a small electrical conductivity/high insulating value in various embodiments.

### Frequency Selection:

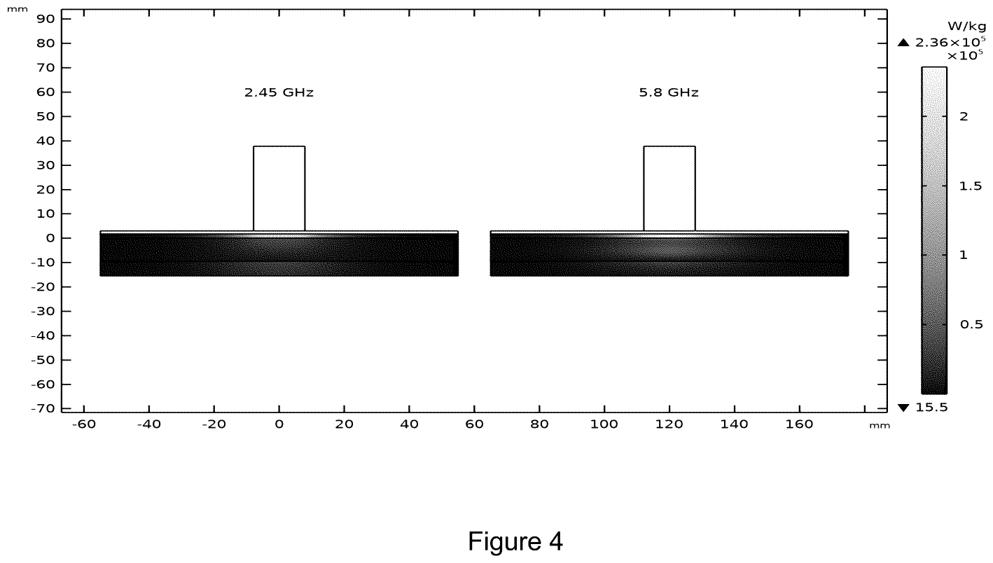

In order to operate the device, a frequency is selected. Portions of the radio frequency spectrum are reserved internationally for non-communication devices, so called ISM bands (industrial, scientific and medical), so that materials processing, microwave ovens etc can operate without regard for telecommunications equipment. According to FCC regulations, Title 47, Chapter I, Subchapter A, Part 15, Section 15.204, Subsection d (https://www.ecfr.gov/cgi-bin/text-idx?SID=ccd706a2c49fd9271106c3228b0615f3&mc=true&node=pt47.1.15&rgn=div5) the available bands are 900 MHz, 2.45 GHz and 5.8 GHz. At 900 MHz, the wavelength in free space is 0.33 m, which is too large for the dimensions of interest here. The frequency selected may range between about 2.45 and about 5.8 GHz. The SAR is plotted in FIG. 4 for the two different frequencies 2.4 GHz (left) and 5.8 GHz (right), for the same total power, 1 kW.

Specific absorption rate (SAR) is a measure of the rate at which energy is absorbed per unit mass by a human body when exposed to a electromagnetic field. The units for SAR are W/kg. As demonstrated in FIGS. 3 and 4, the peak SAR is located near D/F junction, with significant less SAR in tissue underneath the dermis (subcutaneous and muscle in this simulation case). This SAR distribution creates a preferred absorption in dermis and enhanced energy distribution at D/F junction. As a result, a localized selective heating of deep dermis can be achieved non-invasively.

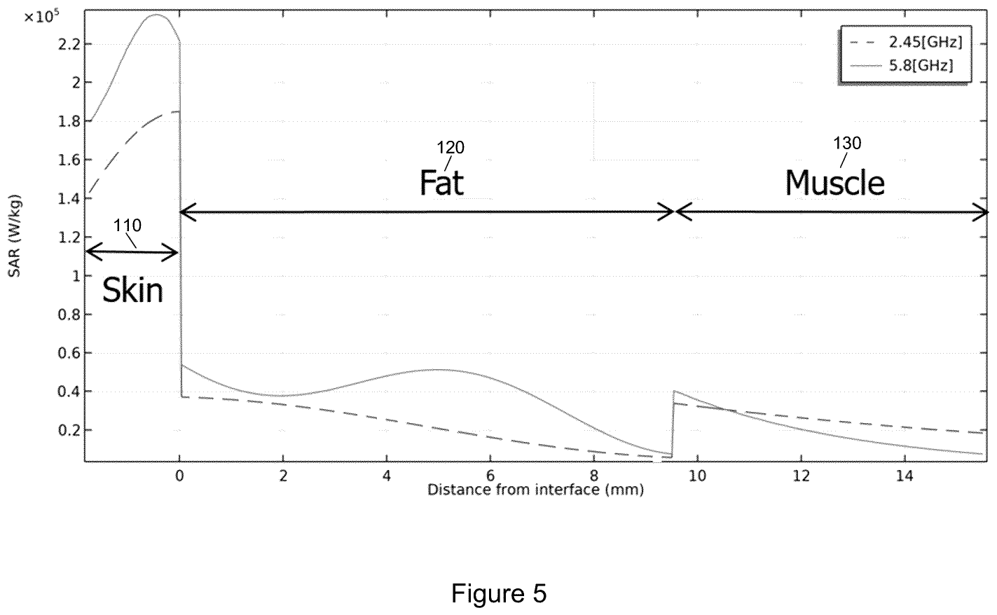

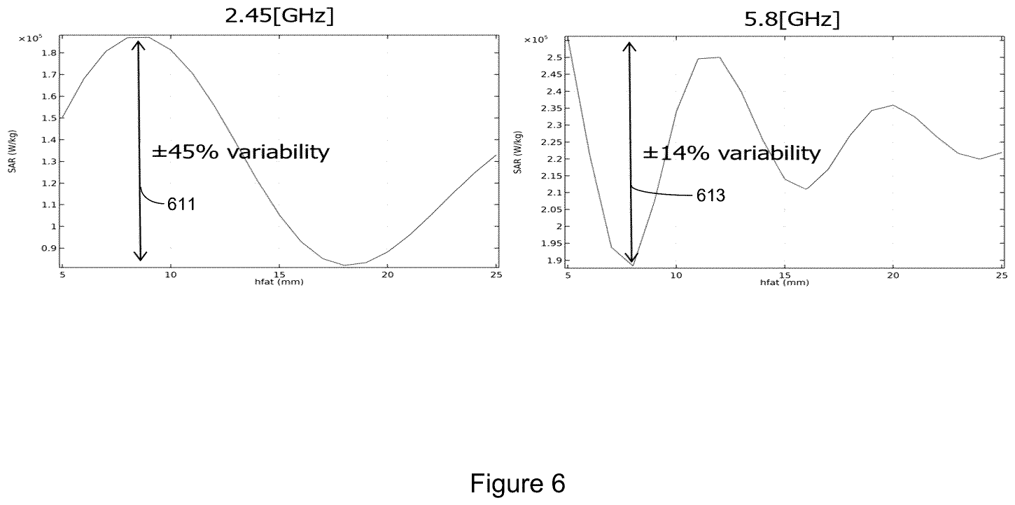

Among the two frequencies, the peak SAR value is higher in the 5.8 GHz case as shown by the values depicted lighter in the greyscale representation that correspond to the upper portion of the SAR legend on the rights side of FIG. 4 (i.e., the right side is at 5.8 GHZ) for the same total power, as shown in FIGS. 4 and 5. A useful difference regarding the variability in the SAR when operating at two frequencies is shown in FIG. 6 below. Specifically, hfat (mm) depicts the thickness of fat between the skin and the muscle, in mm, in FIG. 6. As the fat thickness increases, there is a 45% variability 611 in the SAR when operating at 2.45 GHZ, but only 14% variability 613 when operating at 5.8 GHz. Since the fat content will vary significantly from patient to patient, the higher frequency option is clearly preferable to achieve more repeatable results. Specifically, FIG. 6 depicts the variability in the SAR at the skin/fat interface for different fat thicknesses at two different frequencies where hfat (mm) depicts the thickness of fat between the skin and the muscle, in mm.

### Geometric Dimensions for Exemplary Embodiments

With the frequency locked at 5.8 GHZ, the geometric dimensions can be adjusted. The device can either be operated with a propagating wave, an evanescent wave, or combinations thereof. Operating a device in an evanescent mode is preferable in various embodiments. Evanescent mode is preferable for various reasons that are outlined herewith. In evanescent mode, power is deposited over a small volume, resulting in an increase in device's ability to precisely deliver energy. The precise delivery of energy within a small volume may result in less heat leakage into regions adjacent to the applicator. In turn, improving heat leakage through increased energy delivery precision makes it easier to get to the target temperature at consecutive applications.

Further, in evanescent mode, the power dissipation density will be higher for the same total power, so the skin/fat interface will heat up quicker. This means less total time for which the radiation is applied. An evanescent mode does not propagate electromagnetic radiation into the surrounding environment, because the amplitude of the wave decays exponentially from the source. A propagating mode will radiate energy into the surrounding environment if the applicator was, for example, removed from the patient and pointed elsewhere within the operating room.

### Propagating and Evanescent Modes:

In electromagnetics, an evanescent field, or evanescent wave, is an oscillating electric and/or magnetic field that does not propagate as an electromagnetic wave but whose energy is spatially concentrated near the source (oscillating charges and currents). Even when there is a propagating electromagnetic wave produced (e.g., by a transmitting antenna), one can still identify an evanescent field the component of the electric or magnetic field that cannot be attributed to the propagating wave observed at a distance of many wavelengths (such as the far field of a transmitting antenna). In the case of microwave delivery through a waveguide, a waveguide can be energized in two different ways (propagating and evanescent modes) depending on the height or depth making up the cross-sectional area of the waveguide.

In some embodiments, if the height is greater than half a wavelength, the wave may be classified as propagating, and the wave amplitude follows a cosine along the waveguide, out of the exit aperture. The power flow remains constant along the length of waveguide. The propagation constant of the wave is a real number without a complex component. In contrast, if the height shrinks to less than half a wavelength, the propagation constant becomes an imaginary, a multiple of i, without a real number component. In this case, in which the propagation constant is imaginary, the wave amplitude decays exponentially along the length of the waveguide and the wave may be classified as evanescent. If the length is small enough, or an obstruction is placed in the path of the wave, some of the energy can still escape the waveguide into the surrounding environment before the amplitude exponentially decays to zero.

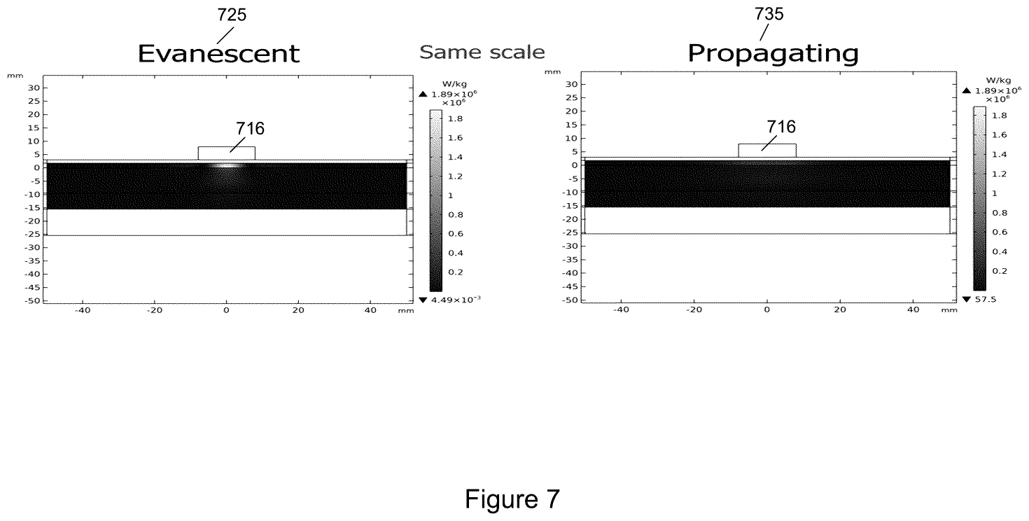

The evanescent mode may be created by either having a short waveguide, such as a waveguide having a length that is below the wavelength, or by adding a restriction to the end of the waveguide. The restriction may be a conductive endface or tissue facing surface that includes one or more apertures. In the following, a short waveguide is used. The length of waveguides in the following simulation are A=1.372 in, a propagating mode, and A=0.343 in, an evanescent mode. The SAR for the evanescent and propagating modes, for the same total input power are shown in FIG. 7 below.

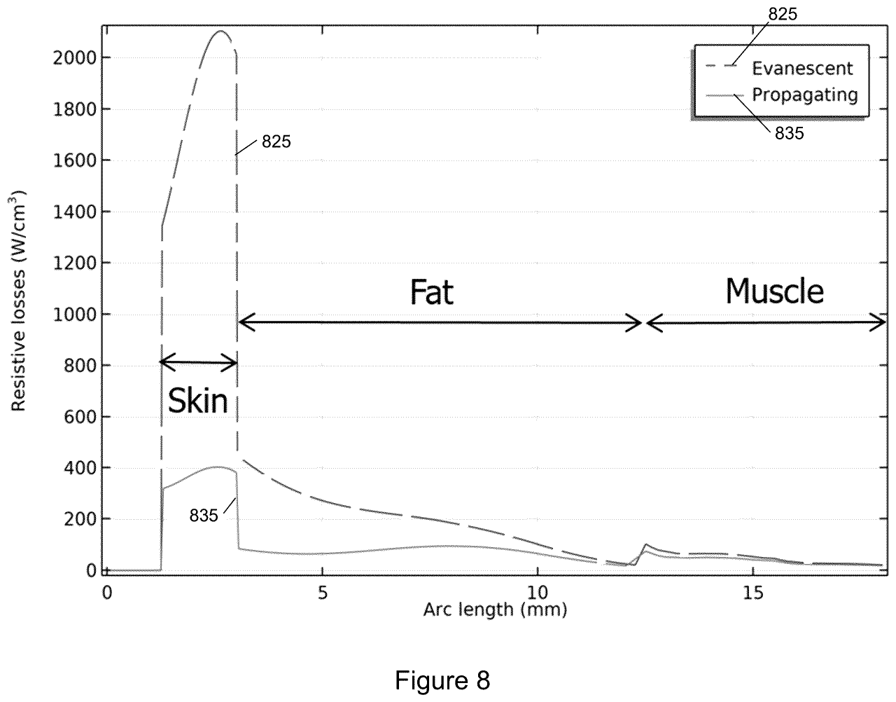

FIG. 7 shows two plots of SAR for the evanescent mode 725 and propagating mode 735 with application 716 of the same total input power. The scale is the same for both plots. The evanescent mode 725 results in a sharper, tighter SAR profile, indicating that the device impact will be more precise than one operating with a propagating mode 735. If an applicator 716 with a short waveguide design is chosen to create evanescent mode the length should be below the wavelength. The minimal length of the wavelength is needed to provide mechanical/thermal strength to avoid damage from the microwave pulse, the dimension should also be sufficiently large for accommodating the size of a feeding pin that couples the power into the waveguide. In some embodiments, the length of the waveguide may be selected from one twentieth of the longest length possible to achieve evanescent mode, or one tenth of the longest length possible to achieve evanescent mode, or one fifth of the longest length possible to achieve evanescent mode. In FIG. 8, the power dissipation is shown at the center of the waveguide aperture with the power dissipation shown as a function of depth for a propagating mode 835 and evanescent mode 825 for the same total power into the system. Because the evanescent mode 825, shown by dotted line, has a narrower power dissipation profile, the magnitude is higher, and weighted slightly more towards the skin/fat interface than the propagating mode 835. The evanescent mode 825 will cause localized heating at a much faster rate than a propagating mode 835, which makes administering the entire procedure easier.

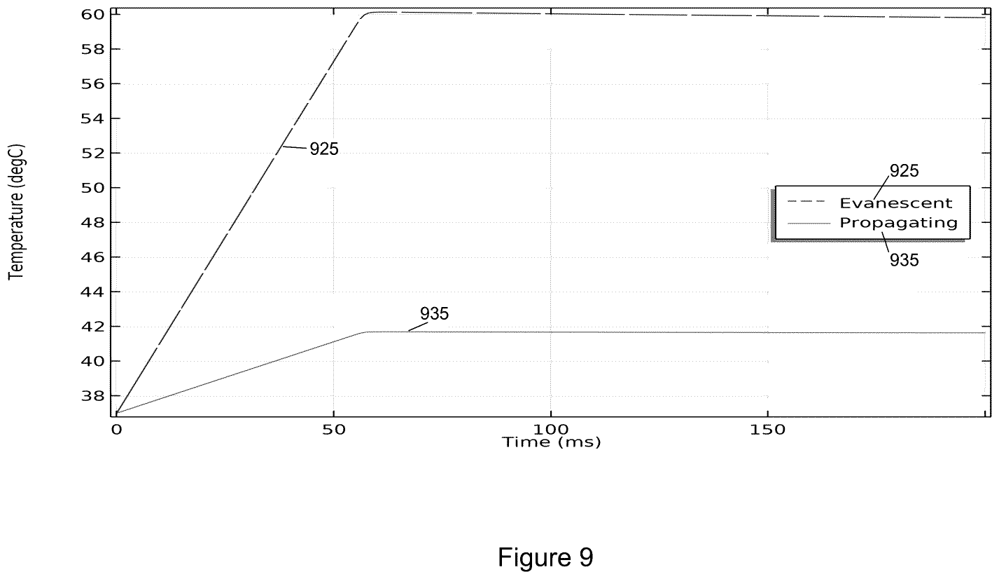

The increase in temperature on the skin/fat interface at the center of the applicator is shown below in FIG. 9 for the same total power input of 1 kW for a propagating wave 935 and an evanescent wave 925.

The evanescent wave 925 can heat the target area to about 60 degrees C. in about 60 ms. Since the temperature increase is almost linear over this time period, the temperature increase may be achieved by delivering about 6 pulses of about 10 ms. In some embodiments, this pulse delivery may be performed with some spacing or off time in between to limit the maximum temperature in the skin itself and mitigate pain. For example, the off time below each pulse can be less than about 100 ms, ideally between about 1 ms to about 10 ms, from about 0.5 ms to about 10 ms, or from about 0.5 ms to about 20 ms. The spread of power dissipation in the propagating mode is very wide, and the temperature rise is only a few degrees over the same time scale.

In some embodiments, to achieve a tissue coagulation temperature of 60° C., the total energy delivered over the about 60 ms time period with 1 kW source is about 60 J. For the benefit of reducing collateral damage and pain management, the heating of the tissue is expected to be limited to less than about 100 ms. In some embodiments, a 600 W microwave source is used to deliver 60 J in this case. A lower power system such as systems with power output in the 100 W range may require much longer heating time (about 600 ms for 100 W system to deliver 60 J energy), which could create more pain and risk of more collateral damage. In various embodiments, an evanescent wave is used to mitigate skin heating during a given cosmetic treatment session.

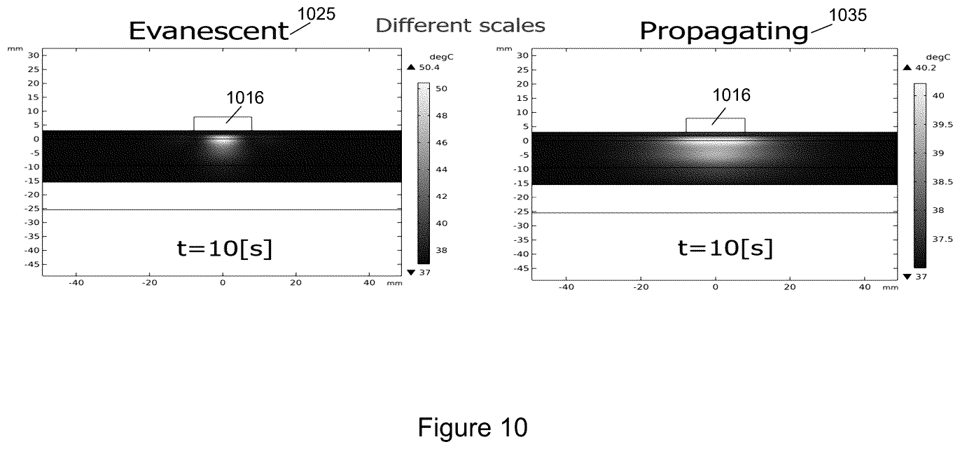

FIG. 10 shows two plots of the temperature distribution in the skin, fat and muscle 10 seconds after a 60 ms application 1016 of 1 kW for evanescent microwaves 1025 and propagating microwaves 1035. The spatial distribution of the temperature indicates that the increase in temperature with an evanescent mode 1025 remains confined to the area directly under the applicator 1016. In various embodiments, this is preferential, because there is little to no excessive heat/thermal energy present prior to the applicator 1016 being moved to an adjacent location on the patient, even after an extended period of time.

Methods for non-invasive skin tightening can employ a microwave source. Suitable microwave sources may have a frequency of from about 300 MHz to 10 GHZ, or from about 2.45 GHz to 5.8 GHz, or at about 5.8 GHz. The microwave source can have a power of at least about 500 Watts, a power of at least about 1 kW, a power of from about 500 Watts to about 1000 Watts, or a power of from about 500 Watts to about 2 KW, where the previously described power may be a peak power. Suitable microwave sources may have pulse widths or pulse durations below 100 ms, below 10 ms, or about Ims. Further, suitable microwave sources may have pulse widths or pulse durations that range from about Ims to about 100 ms, from about Ims to about 50 ms, from about Ims to about 10 ms, from about 10 ms to about 50 ms, or from about 10 ms to about 100 ms. In various embodiments, a microwave generator or multiple microwave generators may be used. The microwave output will be coupled into a handpiece which consists of one or multiple antennae

If it is desirable to operate in a relatively short pulse range, the low power does not achieve the desired temperature for skin tightening treatment. Accordingly, the system operates in a higher power range to achieve the desired temperature range. In some embodiments, a short pulse duration (e.g., 100 ms or shorter), is useful to manage the level of pain experienced by the subject and to avoid unwanted collateral damage in the region of the tissue being treated when the relatively high power level (e.g., at least 500 Watts or about 1 kW) is employed at a frequency of about 5.8 GHz. In various embodiments, a short pulse duration and a high power level operating to deliver an evanescent microwave to a target tissue region are desirable cosmetic treatment parameters.

This approach can be combined with a cooling method which maintains the neighboring tissue temperature below thermal damage threshold, or below heat-pain threshold, or even colder to trigger nerve sensation different from the target area, such as a cool sensation at temperature between approximately 10 degrees C. and body temperature, or a cold sensation at temperature below approximately 10 degrees C.

A given microwave treatment session may also be combined with a mechanical device and may trigger a mechanical nerve sensation to improve patient comfort and mitigate the effects of local heating from microwaves. The embodiment can be any system that generates mechanical force, such as a mechanical roller, a suction chamber etc.

An embodiment that allows the procedure above may include a number of components that might include an microwave energy source, a power modulation system which can adjust output power and pulse width of the energy source, possibly a cooling system to maintain neighboring tissue temperature, such as a chiller with contact cooling or air cooling, and possibly sensors or imaging systems to monitor the treatment parameters, such as reflected energy, tissue temperature, patient thermal sensation etc. The sensors or imaging systems can be in communication with power modulation system to adjust output in real time. The energy sources can be one of any of a number of available microwave sources whose energy can penetrate deep enough to reach the dermal/fat (D/F) junction, or a source whose energy can be focused to the targeted tissue and a scanning system which can overlay the focused energy over the whole treatment area to create a uniform energy distribution or deliver focused energy in an fractionated way.

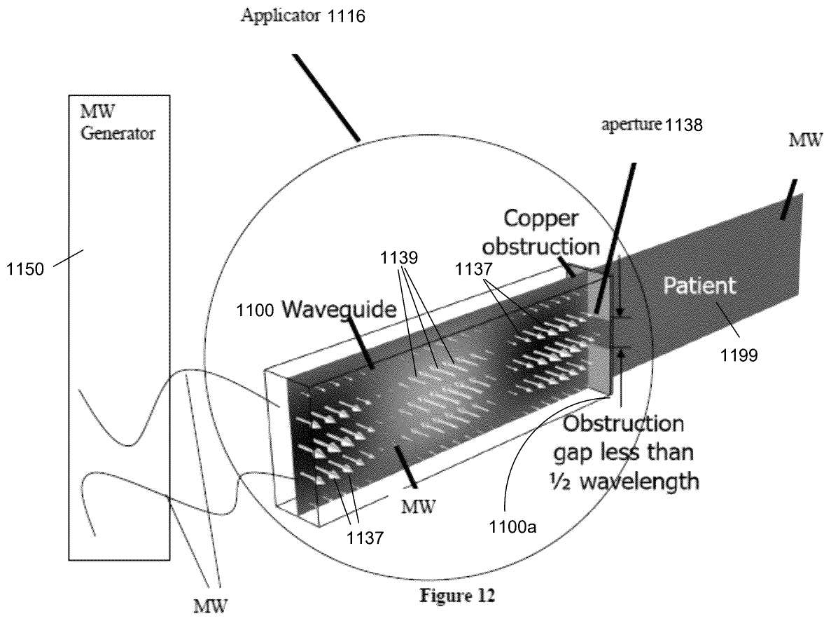

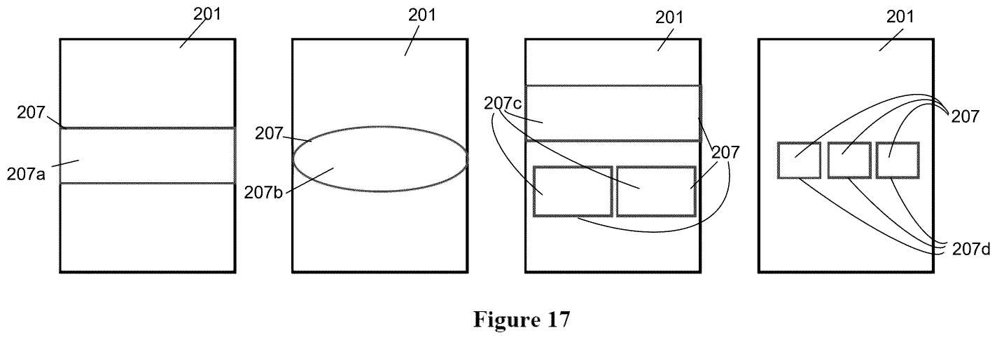

The system may include two major components. A generator that produces the microwave energy at the desired frequency and power level; and a handpiece that is used to apply the energy to the patient. A coaxial cable connects the generator to the handpiece. The coaxial feed is converted into a standing wave via a coaxial to waveguide coupler. The propagating mode results if no obstruction or aperture is used to terminate the waveguide. An obstruction consisting of a highly conductive material, such as copper, is placed at the waveguide aperture, which results in the evanescent mode we are seeking. The opening in the obstruction is typically selected to be less than ½ wavelength, to establish the evanescent mode. In various embodiments, different waveguide shapes with multiple slits, and different combinations of elliptical slits, and other apertures shapes may be used as shown and described in FIGS. 12 and 17.

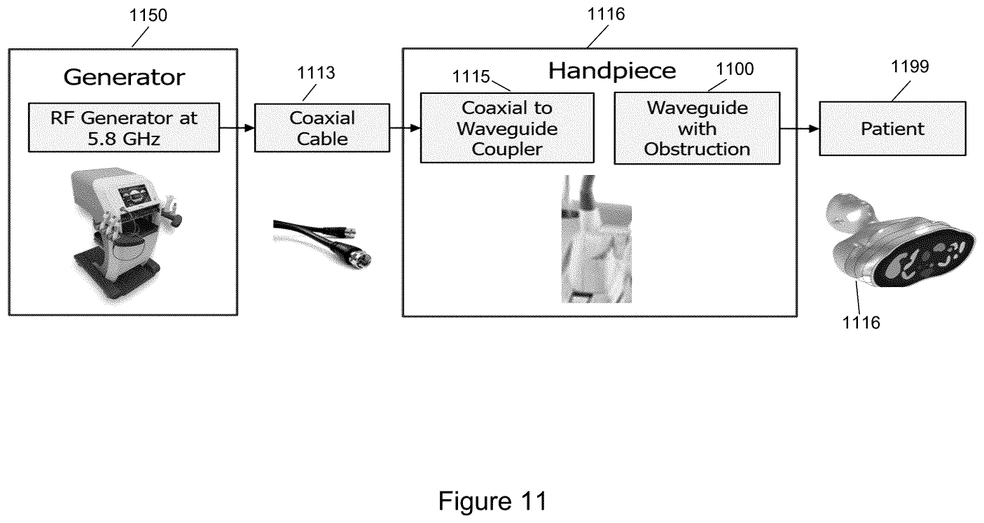

FIG. 11 is a block diagram showing the path of the microwave energy from its source (the generator 1150) to its ultimate destination (the patient 1199). A coaxial cable 1117 or other electrically conductive coupler 1115 may be used to connect the waveguide 1100 of the handpiece 1116 to the microwave generator 1100. A coaxial to waveguide coupler 1115 may be used to connect the coaxial cable 1117 to the waveguide 1100 that includes an endface. In various embodiments, the applicator (also referred to as a handpiece 1116), may include a cooling tissue contacting surface to help reduce pain during cosmetic treatment. The applicator 1116 may also include a vibrating head or elements to facilitate tissue massage and manipulation during treatment. In addition, the applicator 1116 may include one or more surfaces that are transparent to microwaves that are positioned in between the patient 1199 and the waveguide. In some embodiments, a microwave transparent surface may be part of the housing of the applicator 1116.

FIG. 12 is a diagram showing the waveguide 1100, and the portion with the lighter colored arrows represents the electric field, with the lighter white color indicating a negative field 1137 and darker arrows indicating a positive electric field 1139. The direction of the arrow tips (towards viewer or away from viewer) indicate the electric field direction, and each arrow length represents the field amplitude. An end face 1100a or client facing surface obstructs the end of the waveguide 1100 and may also be referred to as an obstruction. In some embodiments, the waveguide endface 1100a or client facing surface defines an aperture 1138 such as a slit or the other aperture configurations shown below in FIG. 17. Referring again to FIG. 12, in some embodiments, the waveguide endface 1100a defines a gap or slit that is less than about ½ wavelength. The microwaves exiting the aperture 1138 continue forward and contact patient tissue 1199 as part of a given cosmetic treatment. In various embodiments, the waveguide 1100, endface 1100a, and aperture 1138 are part of an applicator 1116. In another embodiment, an array of such waveguides 1100 may be energized to create an array of injury zones or uniform injury zone in the patient tissue 1199 as desired.

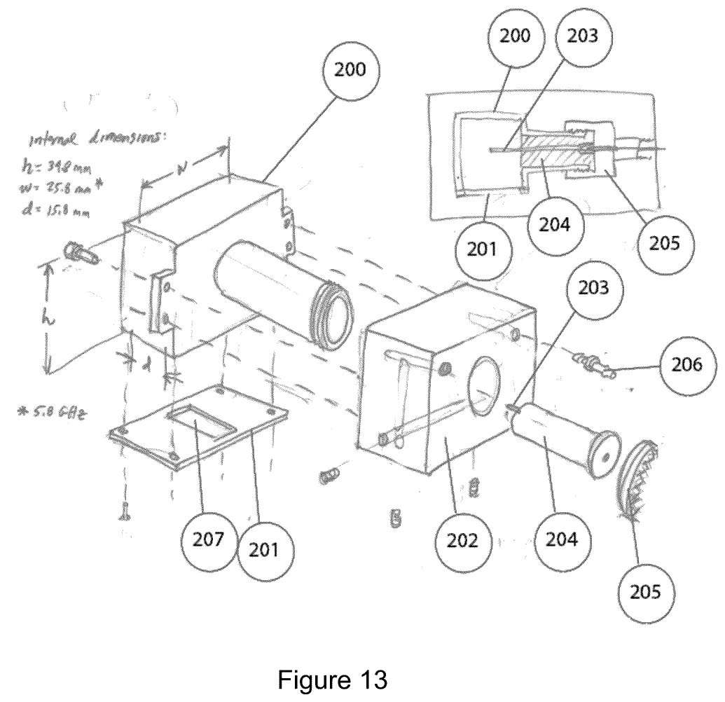

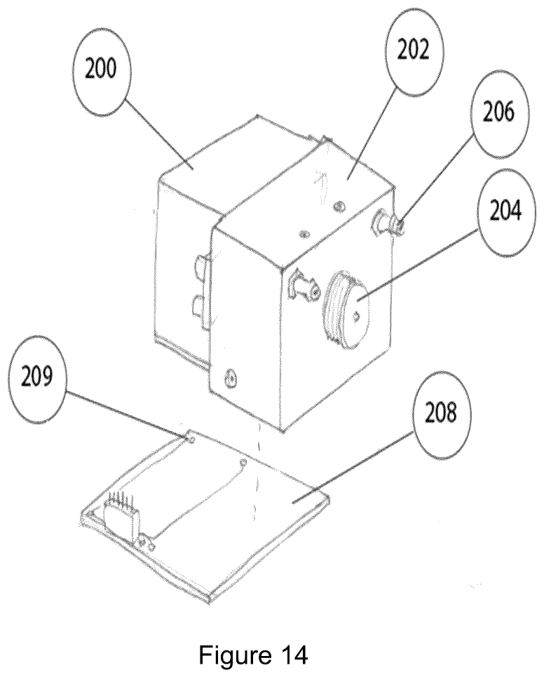

FIGS. 13-16 depict various components of an embodiment of a microwave handpiece. In various embodiments, a waveguide 200 is the metallic chamber that confines the microwave radiation. The waveguide 200 can be machined from metal such as aluminum with dimensions selected to create a standing wave. The height, width and depth of the waveguide 200 are designed in coordination with the microwave wavelength. In one embodiment, for 5.8 GHZ, the dimensions of the waveguide internal dimensions have a height of about 34.8 mm, a width of about 25.8 mm and a depth of about 15.8 mm. The limiting aperture plate 201 generates the evanescent wave and is adjacent to the waveguide's output aperture 207.

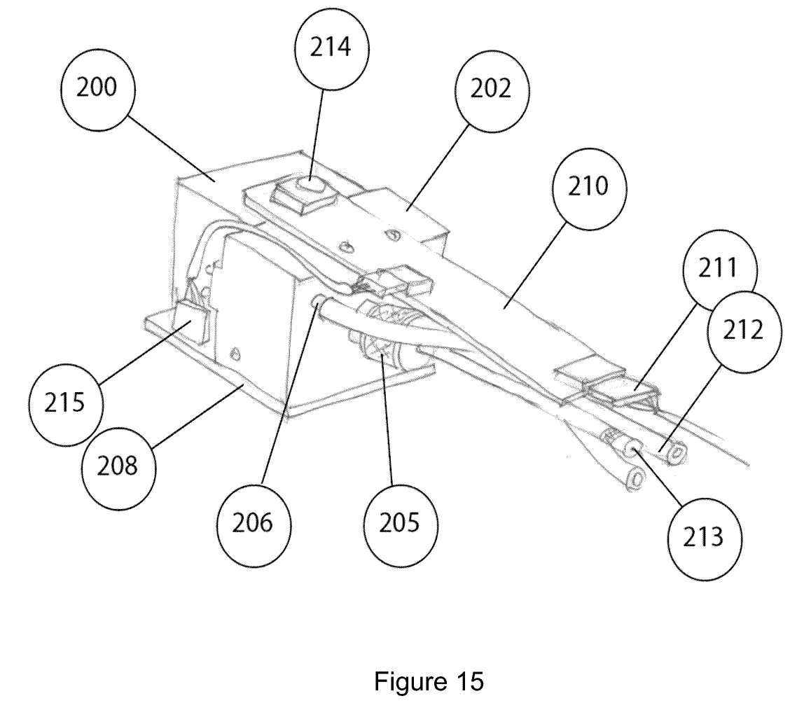

As shown in FIG. 15, the microwave generator is coupled to a coaxial cable 213 and a coaxial connector 205 that couple to the waveguide 200 on a right angle to the waveguide and aperture as shown in the diagram. As shown in FIG. 13, the microwave antenna may include a bare electrode 203 inside a dielectric sheath 204. A cooling block 202 is shown that may include a machined block of copper in one embodiment with machined cooling channels for water to flow. Water or another coolant may be coupled to the cooling block through cooling lines 212 and fittings 206.

Referring again to FIGS. 13-16, in one embodiment, the cooling block 202 is mounted directly adjacent to the waveguide 200 and waveguide aperture plate 201 (where the aperture 207 is disposed) and allows the microwave coupling port to pass through the cooling block. The combined assembly has a flush face whereby a ceramic plate 208 can be thermally epoxied to the machined assembly. The ceramic plate has a thickness selected to not have a significant effect on the microwave output profile but still be mechanically robust enough to be placed on the skin in a stamping fashion and conduct cooling to the underside of the waveguide. In one embodiment, the thickness of the ceramic plate ranges from about 0.25 mm to about 2 mm. In one embodiment, the thickness of the ceramic plate is about 1 mm. This ceramic plate can have temperature measurements and contact sensing 209 to ensure contact is made before the microwave energy is initiated.

Referring now to FIG. 15, the signals from the ceramic PCB/cooling assembly 208 are brought to the main control PCB 210 via connector 215 as shown. The main PCB 210 will have the capability to communicate with the microwave generator. A finger switch 214 or other switches or controls may be used to activate the microwave pulse. Exiting the handpiece 216 and resident in the umbilical 217 are the microwave coaxial cable 213, water lines 212 and the control signals 211. In some embodiments, these may be arranged in other components, housings, or subsystems.

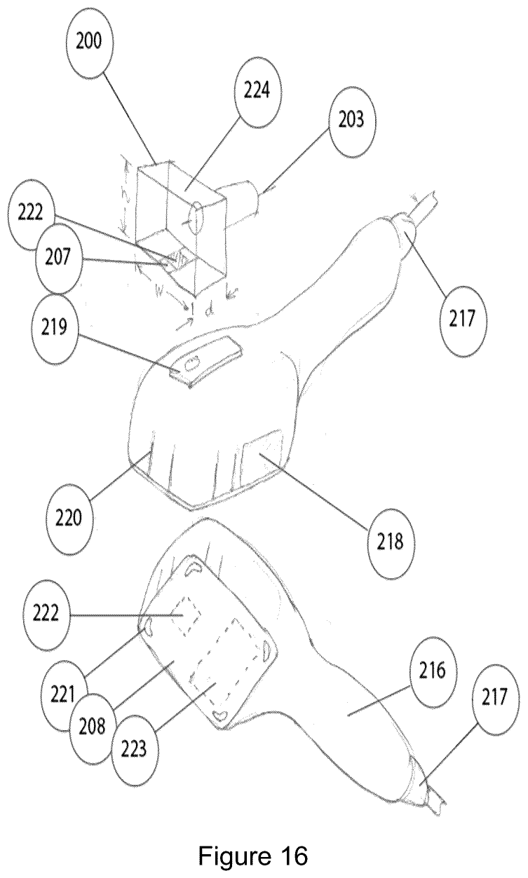

Referring now to FIG. 16, the entire assembly can be housed in a plastic shell 216 or other housing and tethered to or otherwise operably coupled to the microwave generator and console by an umbilical 217. The exterior smooth face of the ceramic applicator may include or position the contact sense electrodes 221. In some embodiments, ensuring full contact with the contact sensors can avoid inconsistent treatment of the target area and enable safe treatment exposure to be delivered to the treatment area. The treatment area 222 may include a portion of the handpiece's footprint. Fiduciary marks 220 on the handpiece body allow the user to approximate or estimate the location of the treatment area when the handpiece is placed on the skin. Another marked area 218 informs the user of the location of the active cooling region is 223. A finger switch button 219 communicates to the finger switch 214 from the outside shell. Various other controls, switches, and inputs may be used in various embodiments.

FIG. 17 shows four waveguide endfaces (e.g., waveguide aperture plates 201) with different apertures 207 defined thereby. The first aperture 207 is a slit 207a that may have a rectangular shape. The second aperture 207 shows a slit 207b that has an elliptical shape. The third endface shows two rectangular apertures combined with a slit defined by the endface 207c. Finally, the fourth endface shows a linear arrangement of rectangular shaped apertures 207d (e.g., three rectangular aperture shapes). Various apertures and combinations of apertures may be used with differing or repeating shapes in various combinations that are suitable for supporting propagating and evanescent microwaves. The applicators or handpieces disclosed herein and variations thereof may be combined with control circuitry to regulate and count the number of uses such that exceeding a treatment limit may be tracked and result in handpiece being directly or remotely deactivated by the supplier or a control system.

Systems and methods utilizing microwave energy to treat a patient's skin (e.g., dermis and hypodermis) or other target tissue at a depth below a tissue surface with microwave energy are described herein. In various aspects, the present teachings can provide a non-invasive, cooled (or uncooled) microwave-based treatment to achieve one or more of sebaceous gland treatment, acne treatment, sweat gland treatment, blood vessel treatment, spider vein treatment, gland damage/deactivation, nerve damage/deactivation, skin tightening (laxity improvement), cellulite treatment apparatus, treatment to remove unwanted hairs, unwanted fat and unwanted vascular lesions by way of non-limiting examples.

In general, the methods and systems disclosed herein may be used to provide various non-medical treatments such as cosmetic treatments, aesthetic treatments, and combinations thereof. Cosmetic treatment of tissue to reduce or prevent excess sweating, removal of unwanted hair, removal of blood vessels and lesions, reducing or preventing acne are all beneficial cosmetic treatments. These and other cosmetic treatments disclosed herein can improve the appearance and well-being of those that suffer with the foregoing conditions and others disclosed herein. In various embodiment, the disclosure relates to methods of controlling transmission of microwave energy such that one or more tissue targets are cosmetically treated to reduce, prevent, reverse, or otherwise cosmetically treat one or more of the unwanted conditions disclosed herein.

It will be appreciated that for clarity, the following discussion will explicate various aspects of embodiments of the applicant's teachings, while omitting certain specific details wherever convenient or appropriate to do so. For example, discussion of like or analogous features in alternative embodiments may be somewhat abbreviated. Well-known ideas or concepts may also for brevity not be discussed in any great detail. The skilled person will recognize that some embodiments of the applicant's teachings may not require certain of the specifically described details in every implementation, which are set forth herein only to provide a thorough understanding of the embodiments. Similarly, it will be apparent that the described embodiments may be susceptible to alteration or variation according to common general knowledge without departing from the scope of the disclosure. The following detailed description of embodiments is not to be regarded as limiting the scope of the applicant's teachings in any manner.

The terms “about” and “substantially identical” as used herein, refer to variations in a numerical quantity that can occur, for example, through measuring or handling procedures in the real world; through inadvertent error in these procedures; through differences/faults in the manufacture of electrical elements; through electrical losses; as well as variations that would be recognized by one skilled in the art as being equivalent so long as such variations do not encompass known values practiced by the prior art. Typically, the term “about” means greater or lesser than the value or range of values stated by 1/10 of the stated value, e.g., +10%. For instance, applying a voltage of about +3V DC to an element can mean a voltage between +2.7V DC and +3.3V DC. Likewise, wherein values are said to be “substantially identical,” the values may differ by up to 5%. Whether or not modified by the term “about” or “substantially” identical, quantitative values recited in the claims include equivalents to the recited values, e.g., variations in the numerical quantity of such values that can occur, but would be recognized to be equivalents by a person skilled in the art.

Moreover, nothing disclosed herein is intended to be dedicated to the public regardless of whether such disclosure is explicitly recited in the claims. To aid the Patent Office and any readers of any patent issued on this application in interpreting the claims appended hereto or otherwise presented throughout prosecution of this or any continuing patent application, applicants wish to note that they do not intend any claimed feature to be construed under or otherwise to invoke the provisions of 35 USC 112 (f), unless the phrase “means for” or “step for” is explicitly used in the particular claim.

All of the drawings submitted herewith include one or more ornamental features and views, each of which include solid lines any of which also incorporate and correspond to and provide support for dotted lines and alternatively, each of which include dotted lines any of which also incorporate and correspond to and provide support for solid lines.

The use of the terms “include,” “includes,” “including,” “have,” “has,” or “having” should be generally understood as open-ended and non-limiting unless specifically stated otherwise.

The use of the singular herein includes the plural (and vice versa) unless specifically stated otherwise. Moreover, the singular forms “a,” “an,” and “the” include plural forms unless the context clearly dictates otherwise. In addition, where the use of the term “about” is before a quantitative value, the present teachings also include the specific quantitative value itself, unless specifically stated otherwise.

It should be understood that the order of steps or order for performing certain actions is immaterial so long as the present teachings remain operable. Moreover, two or more steps or actions may be conducted simultaneously.

Where a range or list of values is provided, each intervening value between the upper and lower limits of that range or list of values is individually contemplated and is encompassed within the disclosure as if each value were specifically enumerated herein. In addition, smaller ranges between and including the upper and lower limits of a given range are contemplated and encompassed within the disclosure. The listing of exemplary values or ranges is not a disclaimer of other values or ranges between and including the upper and lower limits of a given range.

It should be appreciated that numerous changes can be made to the disclosed embodiments without departing from the scope of the present teachings. While the foregoing figures and examples refer to specific elements, this is intended to be by way of example and illustration only and not by way of limitation. It should be appreciated by the person skilled in the art that various changes can be made in form and details to the disclosed embodiments without departing from the scope of the teachings encompassed by the appended claims.

1. A non-invasive skin tightening system comprising:
an applicator comprising a first surface, the first surface defining one or more apertures;
a waveguide in communication with the first surface and positioned to direct microwaves through the one or more apertures; and
a microwave generator having a peak power, a pulse width, and a microwave frequency.

2. The system of claim 1, wherein the microwave frequency ranges from about 300 MHz to about 10 GHz, optionally wherein the microwave frequency is about 5.8 GHz.

3. The system of claim 1, wherein the microwave frequency ranges from about 2 GHz to about 6 GHz.

4. The system of claim 1, wherein the one or more apertures is a slit, wherein the slit has a length and a width, wherein the length ranges from about 5 mm to about 15 mm and wherein the width ranges from about 12.5 mm to about 18 mm.

5. The system of claim 1, wherein a length of waveguide ranges from about 28 mm to about 38 mm, wherein width of waveguide ranges from about 12.5 mm to about 18 mm, and wherein height of waveguide is fixed to an integer multiple of half wavelengths.

6. The system of claim 1, wherein the peak power has a range, wherein the range is greater than or equal to about 1 kW, optionally wherein the pulse width ranges from about 0.5 ms to about 100 ms.

7. The system of claim 1, wherein the peak power ranges from about 0.5 kW to about 10 kW.

8. The system of claim 1 further comprising a control system, wherein the control system is in electrical communication with the microwave generator, wherein the control system is operable to cause the microwave generator to generate propagating microwaves, evanescent microwaves, and combinations thereof.

9. The system of claim 8, wherein control system is operable to generate constructive interference of microwaves from the microwave generator at dermal and fat junction and to generate one or more confined thermal heating zones near the dermal and fat junction at one or more target tissue regions.

10. The system of claim 2, wherein the one or more apertures comprise one or more shapes configured to support propagating or evanescent microwaves, wherein the microwave generator has a peak power that ranges from about 1 kW to about 20 kW.

11. The system of claim 9, wherein control system regulates the microwave generator to cause collagen coagulation at one or more target tissue regions.

12. The system of claim 5, wherein the integer multiple of half wavelengths is about N (25.85) mm, wherein N is a positive integer.

13. The system of claim 12, wherein the waveguide has an internal dimension that comprises one or more of a height of about 34.8 mm, a width of about 25.8 mm and a depth of about 15.8 mm.

14. A non-invasive skin tightening system comprising:
an applicator comprising a first surface, the first surface defining one or more apertures;
a waveguide in communication with the first surface and positioned to generate evanescent waves; and
a microwave generator having a peak power, a pulse width, and a microwave frequency, wherein the microwave frequency ranges from about 300 MHz to about 10 GHz, wherein the waveguide has an internal dimension that comprises one or more of a height of about 34.8 mm, a width of about 25.8 mm and a depth of about 15.8 mm.

15. The system of claim 14 further comprising a cooling system in communication with a treatment surface of the applicator and or the microwave generator or waveguide.

16. The system of claim 14, wherein the microwave frequency ranges from about 2 GHz to about 6 GHz.

17. The system of claim 15, wherein the peak power ranges from about 0.5 kW to about 10 KW, optionally wherein the pulse width ranges from about 0.5 ms to about 100 ms.

18. The system of claim 14, wherein the pulse width ranges from about 0.5 ms to about 100 ms.

19. A non-invasive cosmetic method for tightening skin, the method comprising:
applying to a skin surface an applicator surface defining one or more apertures; and
generating evanescent waves with a waveguide in communication with the applicator surface, the waveguide emitting a microwave having a peak power, a pulse width, and a microwave frequency.

20. The method of claim 19, wherein the peak power ranges from about 0.5 kW to about 10 KW, wherein the pulse width ranges from about 0.5 ms to about 100 ms, wherein the microwave frequency ranges from about 300 MHz to about 10 GHz.
